(19) United States
(12) Patent Application Publication
(10) Pub. No.: US 2011/0184265 A1
(43) Pub. Date: Jul. 28, 2011

(54) Method and Apparatus for Providing Notification in Analyte Monitoring Systems

(75) Inventor: Gary Alan Hayter, Oakland, CA (US)

(73) Assignee: Abbott Diabetes Care Inc., Alameda, CA (US)

(21) Appl. No.: 13/011,897

(22) Filed: Jan. 22, 2011

Related U.S. Application Data
(60) Provisional application No. 61/297,615, filed on Jan. 22, 2010.

(51) Int. Cl. A61B 5/145

(52) U.S. Cl. 600/347

(57) ABSTRACT

An analyte monitoring system for determining an analyte concentration of a biofluid upon user command and adapted to determine rate of change of the analyte concentration in addition to the real time analyte concentration and to output an alarm notification upon an anticipated physiological condition determined by projected analyte levels is provided. Methods, devices and kits are also provided.

### RELATED APPLICATION

The present application claims the benefit of US provisional application No. 61/297,615 filed Jan. 22, 2010, entitled “Projected Glucose-On-Demand Alarm”, the disclosure of which is incorporated herein by reference for all purposes.

### BACKGROUND

Diabetes mellitus is an incurable chronic disease in which the body does not produce or properly utilize insulin. Insulin is a hormone produced by the pancreas that regulates blood glucose. In particular, when blood glucose levels rise, e.g., after a meal, insulin lowers the blood glucose levels by facilitating blood glucose to move from the blood into the body cells. Thus, when the pancreas does not produce sufficient insulin (a condition known as Type I Diabetes) or does not properly utilize insulin (a condition known as Type II Diabetes), the blood glucose remains in the blood resulting in hyperglycemia or abnormally high blood sugar levels.

People suffering from diabetes often experience long-term complications. Some of these complications include blindness, kidney failure, and nerve damage. Additionally, diabetes is a factor in accelerating cardiovascular diseases such as atherosclerosis (hardening of the arteries), which often leads stroke, coronary heart disease, and other diseases, which can be life threatening.

The severity of the complications caused by both persistent high glucose levels and blood glucose level fluctuations has provided the impetus to develop diabetes management systems and treatment plans. In this regard, diabetes management plans historically included multiple daily testing of blood glucose levels typically by a finger-stick to draw and test blood. The disadvantage with finger-stick management of diabetes is that the user becomes aware of his blood glucose level only when he performs the finger-stick. Thus, blood glucose trends and blood glucose snapshots over a period of time was unknowable. More recently, diabetes management has included the implementation of glucose monitoring systems. Glucose monitoring systems have the capability to continuously or periodically monitor a user's glucose levels. Thus, such systems have the ability to illustrate not only present blood glucose levels but also provide snapshot of glucose levels and fluctuations over a period of time.

Glucose monitoring systems also have the capability to output alarm notifications, such as an audible alarm, to alert the user to a condition that may require medical attention. Such alarms are usually triggered when the blood glucose level of a patient exceed a preset glucose level threshold. Some glucose monitoring systems also include projected alarms that warn the user of an impending high or low glucose level.

The method of calculating the projected alarms varies according to the glucose monitoring system being used. For example, some glucose monitoring systems use the present glucose level and its rate of change (slope) to make a straight-line extrapolation of the glucose value at times in the future. If the glucose value is projected to be above or below a certain threshold within some time, the projected alarm is sounded. The user experience is very much affected by the frequency of the projected alarms.

### INCORPORATION BY REFERENCE

Patents, applications and/or publications described herein, including the following patents, applications and/or publications are incorporated herein by reference for all purposes: U.S. Pat. Nos. 4,545,382; 4,711,245; 5,262,035; 5,262,305; 5,264,104; 5,320,715; 5,356,786; 5,509,410; 5,543,326; 5,593,852; 5,601,435; 5,628,890; 5,820,551; 5,822,715; 5,899,855; 5,918,603; 6,071,391; 6,103,033; 6,120,676; 6,121,009; 6,134,461; 6,143,164; 6,144,837; 6,161,095; 6,175,752; 6,270,455; 6,284,478; 6,299,757; 6,338,790; 6,377,894; 6,461,496; 6,503,381; 6,514,460; 6,514,718; 6,540,891; 6,560,471; 6,579,690; 6,591,125; 6,592,745; 6,600,997; 6,605,200; 6,605,201; 6,616,819; 6,618,934; 6,650,471; 6,654,625; 6,676,816; 6,730,200; 6,736,957; 6,746,582; . 6,749,740; 6,764,581; 6,773,671; 6,881,551; 6,893,545; 6,932,892; 6,932,894; 6,942,518; 7,041,468; 7,167,818; 7,299,082; and 7,866,026; U.S. Published Application Nos. 2004/0186365; 2005/0182306; 2006/0025662; 2006/0091006; 2007/0056858; 2007/0068807; 2007/0095661; 2007/0108048; 2007/0199818; 2007/0227911; 2007/0233013; 2008/0066305; 2008/0081977; 2008/0102441; 2008/0148873; 2008/0161666; 2008/0267823; 2009/0054748; 2009/0247857; 2009/0294277; 2010/0081909; 2010/0198034; 2010/0213057; 2010/0230285; 2010/0313105; 2010/0326842; and 2010/0324392; U.S. patent application Ser. Nos. 12/807,278; 12/842,013; and 12/871,901; and U.S. Provisional Application Nos. 61/238,646; 61/246,825; 61/247,516; 61/249,535; 61/317,243; 61/345,562; and 61/361,374.

### SUMMARY

An analyte monitoring system in certain embodiments includes an analyte sensor adapted to measure one or more analyte concentrations present in a bodily fluid of a user and to generate signals corresponding an analyte concentration, sensor electronics in signal communication with the analyte sensor and configured to transmit the signals corresponding to the measured analyte concentration in response to a command, and a receiver configured to generate and transmit the command to the sensor electronics, and in response to the transmitted command, to receive the signals corresponding to the measured analyte concentration from the sensor electronics, the received signals including multiple data points corresponding to the measured analyte concentration over a predetermined time period, the receiver including a display configured to output the received one or more signals from the sensor electronics, the receiver configured to determine a real time analyte concentration level and a current status of an anticipated physiological condition based on the received multiple data points, and further where the receiver is configured to output a notification associated with the determined current status of the anticipated physiological condition, where the sensor electronics transmits the multiple data points corresponding to the measured analyte concentration over the predetermined time period in a single data transmission to the receiver in response to the received command, and further where the receiver outputs an indication of the determined real time analyte concentration level and an indication of the determined current status of the anticipated physiological condition on the display.

In one aspect of the disclosure, a medical system includes an analyte monitoring system that provides user analyte information based on a user initiated command. The medical system can include an operative component that is adapted to measure one or more analyte concentrations present in a bodily fluid of a user and a processor adapted to transmit the one or more analyte concentrations to a receiver when commanded by a user. The receiver of the system can be configured to issue a notification of an anticipated physiological condition based on the received one or more signals. In one embodiment, a notification of an anticipated condition is issued by the receiver upon user command.

The medical system can also include a processor configured to transmit data comprising a plurality of analyte concentrations measured over a period of time to the receiver when commanded by the user. The receiver can be adapted to process the data comprising the plurality of analyte concentrations to determine trend information. The trend information can be utilized by the receiver to project a future analyte concentration level. In another aspect, the receiver can be adapted to issue a notification based on the projected analyte concentration level.

In another embodiment, the receiver can be configured to receive analyte concentration levels over time and store the data comprising the plurality of analyte concentrations. The receiver can be adapted to process the stored data to determine trend information. In another aspect, the receiver can be adapted to project a future analyte concentration level based on the stored plurality of analyte concentrations or trend information derived therefrom.

The notification issued by the receiver can be a visual indicator displayed by the receiver, an audible indicator or a tactile indicator. Some non limiting examples of a visual indicator include a popup screen, a displayed icon, or illuminated light sources. Some non-limiting examples of an audio notification include a beep or recorded voice. Some non-limiting examples of a tactile indicator include vibratory messaging or mild electric shock. In some embodiments, the notification can be enabled or disabled by the user. Additionally, the mode of notification can be changed or programmed by the user.

In some instances, the anticipated physiological condition can for example be an elevated analyte concentration or a depressed analyte concentration. For example, hypoglycemia or hyperglycemia can be an anticipated physiological condition.

The medical system includes an on-demand analyte monitoring system. In one embodiment, the processor is a transceiver configured for bidirectional communication. In another embodiment, the receiver is configured for bidirectional communication. The transceiver and receiver components can communicate via a radiofrequency link upon user command. For example, the transceiver and receiver can include radiofrequency identification. The user command can include placing the receiver and transceiver components in close proximity. For example, the close proximity can be established by a distance of less than about three inches. Upon bringing the receiver and transceiver in close proximity the receiver can send a signal to the transceiver to command transmission of one or more signals relating to one or more analyte measurements. In another aspect, the transceiver does not require a battery. Instead, the receiver can be adapted to power the transceiver while in close proximity to the transceiver. In this regard, the transceiver can have a size and configuration for easy wear and comfort for a user.

### BRIEF DESCRIPTION OF THE FIGURES

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part. Like reference numerals used in different figures denote like components or process steps. Reference numerals that differ only in the hundreds or thousands place from reference numerals in earlier figures refer (unless the context requires otherwise) to components or process steps that may be adapted from the corresponding component or process step in the prior figure.

FIG. 1 is a schematic illustration of the components of an analyte monitoring system in accordance with embodiments of the present disclosure;

FIG. 2 is a block diagram of the transmitter device of the analyte monitoring system shown in FIG. 1 in accordance with embodiments of the present disclosure;

FIG. 3 is a block diagram of the receiver device of the analyte monitoring system shown in FIG. 1 in accordance with embodiments of the present disclosure;

FIGS. 4A-4B illustrate a perspective view and a cross sectional view, respectively, of an analyte sensor in accordance with embodiments of the present disclosure;

FIG. 5 is a block diagram of the transmitter device of the analyte monitoring t system shown in FIG. 1 in accordance with embodiments of the present disclosure;

FIG. 6 illustrates a receiver in accordance with embodiments of the present disclosure;

FIGS. 7 and 8 illustrate displays of the user interface in accordance with embodiments of the present disclosure; and

FIGS. 9 and 10 illustrate displays of the user interface in accordance with embodiments of the present disclosure.

### DETAILED DESCRIPTION

It should be understood, in connection with the following description, that the subject matter is not limited to particular embodiments described, as the particular embodiments of the subject matter may of course vary. It is also to be understood that the terminology used herein is for the purpose of describing particular embodiments only, and is not intended to be limiting, since the scope of the disclosed subject matter will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter.

Every range stated is also intended to specifically disclose each and every “subrange” of the stated range. That is, each and every range smaller than the outside range specified by the outside upper and outside lower limits given for a range, whose upper and lower limits are any tenth of the unit of the outside lower limit within the range from said outside lower limit to said outside upper limit (unless the context clearly dictates otherwise), is also to be understood as encompassed within the disclosed subject matter, subject to any specifically excluded range or limit within the stated range. Where a range is stated by specifying one or both of an upper and lower limit, ranges excluding either or both of those stated limits, or including one or both of them, are also encompassed within the disclosed subject matter, regardless of whether or not words such as “from”, “to”, “through”, or “including” are or are not used in describing the range.

Unless defined otherwise, all technical and scientific terms used herein have the same meaning as commonly understood by one of ordinary skill in the art to which this disclosed subject matter belongs. Although any methods and materials similar or equivalent to those described herein can also be used in the practice or testing of the present disclosed subject matter, this disclosure may specifically mention certain exemplary methods and materials.

All publications mentioned in this disclosure are, unless otherwise specified, incorporated herein by reference for all purposes, including without limitation to disclose and describe the methods and/or materials in connection with which the publications are cited.

The publications discussed herein are provided solely for their disclosure prior to the filing date of the present application. Nothing herein is to be construed as an admission that the present disclosed subject matter is not entitled to antedate such publication by virtue of prior invention. Further, the dates of publication provided may be different from the actual publication dates, which may need to be independently confirmed.

As used herein and in the appended claims, the singular forms “a”, “an”, and “the” include plural referents unless the context clearly dictates otherwise.

Nothing contained in the Abstract or the Summary should be understood as limiting the scope of the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosed subject matter. Any recited method can be carried out in the order of events recited, or in any other order which is logically possible. Reference to a singular item, includes the possibility that there are plural of the same item present. When two or more items (for example, elements or processes) are referenced by an alternative “or”, this indicates that either could be present separately or any combination of them could be present together except where the presence of one necessarily excludes the other or others.

Certain classes of analyte monitors are provided in small, lightweight, battery-powered and electronically-controlled systems. Such a system may be configured to detect signals indicative of in vivo analyte levels using an electrochemical sensor, and to process and/or collect such signals. In some embodiments, the portion of the system that performs this initial processing may be configured to transmit the data to another unit for further collection and/or processing. Such transmission may be effected, for example, via a wired connection, such as electrical contacts or a cable, or via a wireless connection, such as an infrared (IR) or radio frequency (RF) connection.

Certain analyte monitoring systems for in vivo measurement employ a sensor that measures analyte levels in interstitial fluids within the subject's tissue. These may be inserted partially through the skin or entirely under the skin. A sensor in such a system may operate as an electrochemical cell. Such a sensor may use any of a variety of electrode configurations, such as a three-electrode configuration (e.g., with “working”, “reference” and “counter” electrodes), driven by a controlled potential (potentiostat) analog circuit, a two-electrode system configuration (e.g., with working and counter electrodes and a resistance), which may be self-biasing and/or self-powered, and/or other configurations. Other configurations may be used, for example, sensor having multiple working and/or counter electrodes.

In certain systems, the analyte sensor is in communication with a processor/transmitter unit; the term “transmitter unit” or “transmitter device” as used in this disclosure refers to such a combination of an analyte sensor with such a data processor/transmitter. Certain embodiments are modular. The transmitter device may be separately provided as a physically distinct assembly, and configured to transmit the analyte levels detected by the sensor over a communication link to a receiver/monitor unit, referred to in this disclosure as a “receiver unit” or “receiver device”, or in some contexts, depending on the usage, as a “meter”.

The receiver unit may perform data analysis, evaluation, calculation, and so on, on the received analyte levels to generate information pertaining to the monitored analyte levels. The receiver unit may incorporate a display screen, which can be used, for example, to display measured analyte levels. It is also useful for a user of an analyte monitor to be able to see trend indications (including the magnitude and direction of any ongoing trend), and such data may be displayed as well, either numerically, or by a visual indicator, such as an arrow that may vary in visual attributes, such as size, shape, color, animation, or direction. The receiver unit may further incorporate a blood glucose test strip port and related electronics in order to be able to make discrete (e.g., in vitro blood glucose (BG)) measurements.

The modularity of these systems may vary. In some embodiments the sensor is attachable and detachable from the transmitter, and the sensor may be disposable and the transmitter reusable. In other embodiments, the sensor and transmitter may be provided as an integrated package, which may be disposable.

To provide flexibility in analyte sensor manufacturing and/or design, it may be desirable for the transmitter device to accommodate a substantial range of analyte sensor sensitivities. Methods and systems for measuring sensor sensitivity are desirable in such cases, so that the analyte monitor may be accurately calibrated.

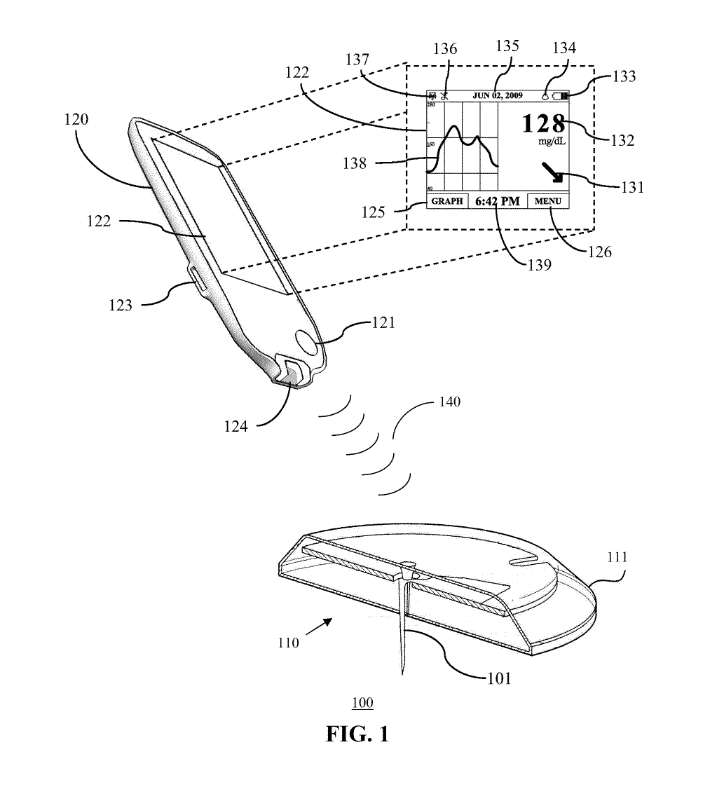

FIG. 1 shows an embodiment of an analyte monitoring system 100. In such a system, transmitter device 110 (shown in cross-section in FIG. 1) may comprise an analyte sensor 101 and a transmitter (with associated electronics 111). Receiver unit 120 may also be provided. In the embodiment shown, transmitter device 110 and receiver 120 communicate via connection 140 (in this embodiment, e.g., a wireless radiofrequency (RF) connection). Also shown on receiver 120 is a port 124 for performing a reference analyte measurement, e.g., reading a blood glucose test strip, an input button 121 (which may be used to power on and off the receiver 120), and a data port 123, such as a USB port. In certain embodiments, receiver 120 may include only some of the mentioned features, or may include additional features, such as additional input or output features.

Still referring to FIG. 1, receiver 120 is shown with a display 122. Display 122 may be capable of displaying a variety of screens, graphs and indicators. For example, as shown, display 122 includes a menu button 126, a graph button 125, the time 139, the date 135, a graphical output 138, a numerical output 132, an directional arrow output 131, a batter icon 133, a calibration icon 134, a sound or vibrate icon 136 and a wireless connectivity icon 137. It is contemplated that a variety of displays, icons, and menus may be provided on display 122 of receiver 120.

Analyte monitoring system 100 may be used to monitor levels of a wide variety of analytes. Analytes that may be monitored include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored.

In certain embodiments, system 100 may be a continuous analyte monitor (e.g., CGM), and accordingly, operate in a mode in which the RF communications has sufficient range to support a flow of data from transmitter device 110 to receiver unit 120. In some embodiments, data may be transmitted on a periodic basis, such as once a minute, once every 30 seconds, once every five minutes, etc. In other embodiments, transmitter device 110 may further comprise local memory in which it may record “logged data” collected over a period of time and provide the accumulated data to receiver unit 120 from time-to-time. Logged data may be accumulated over the last 10-15 minutes, over several hours, or over several days, etc. In some embodiments, a separate data logging unit may be provided to acquire periodically transmitted data from transmitter device 110. Depending on the manner of interaction of the transmitter device and the receiver (and/or data logger) unit, these embodiments may be considered CGMs as well for purposes of this disclosure.

Detailed description of continuous analyte monitoring devices can be found in U.S. patent application Ser. No. 12/873,298, filed Aug. 31, 2010, incorporated by reference herein in its entirety, for all purposes. Additionally, commonly assigned U.S. patent application Ser. No. 12/873,298, filed Aug. 31, 2010 and U.S. patent application Ser. No. 12/807,278 filed Aug. 31, 2010 describe systems (e.g., system 100) including an “on-demand” analyte monitor where analyte measurements are either transmitted from the sensor electronics to the receiver upon request by the user, the disclosures of each of which are hereby each incorporated by reference in their entirety herein for all purposes.

In certain embodiments, in an on-demand monitor, analyte data can be transferred at any time, i.e., “on-demand,” e.g., by placing a receiver or meter device in close proximity with the a transmitter device and initiating a data transfer, either over a wired connection, or wirelessly by various means, including, for example various RF-carried encodings and protocols and IR links. In some embodiments, the data transferred to the receiver unit is obtained instantaneously by the analyte sensor upon receipt of a request of a user. In some embodiments, the data transferred to the receiver unit is the most recent analyte data obtained by the sensor. Such data may be combined with analyte data obtained from physical samples, such as from a blood glucose test strip, or with event data, e.g., eating times and exercise, provided to the receiver or meter device by the user.

On-demand monitors may be configured to reduce the size and cost and increase the convenience of use of transmitter device 110, which in these embodiments is the on-body component. Low power operation may be a major consideration. In this regard, in certain embodiments, transmitter device 110 may be powered without an internal battery or power source, or alternatively, by a power source, such as an external power source inductively coupled to the transmitter, or by an induction generator, powered by user movement. In other embodiments, a small battery may be provided.

In some embodiments, an internal memory may be provided to store measured values for transmission when an on-demand communications session is established with receiver 120. In still other embodiments, an analog circuit, powered by sensor 101, may be incorporated to provide averaged, delayed and/or sequenced measurement data, which in battery-less embodiments could be used in lieu of digitally stored system measurements. In addition, digitally processed and/or stored data on-board transmitter device 110 may be combined with various analog signal processing or pre-processing techniques.

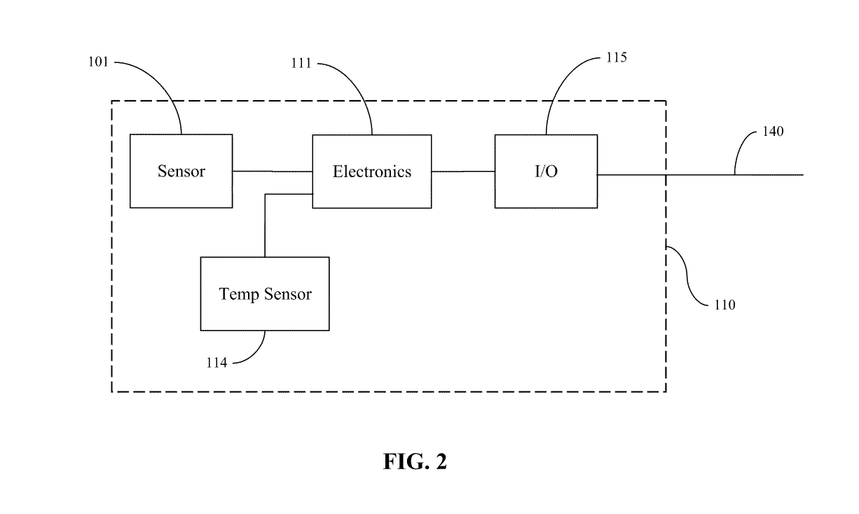

Referring to FIG. 2, transmitter unit 110 may comprise sensor 101, temperature sensors 114, electronics 111 coupled to sensor 101 and temperature sensor 114, and transmitter input-output (I/O) components 115. In one embodiment, the transmitter device 110 is physically coupled to the sensor 101 so that both devices are positioned on the user's body, with at least a portion of sensor 101 positioned transcutaneously under the skin layer of the user. The transmitter device 110 may perform data processing such as filtering and encoding on data signals, each of which corresponds to a sampled analyte level of the user, for transmission to the receiver unit 120 (FIG. 1) via the communication link 140.

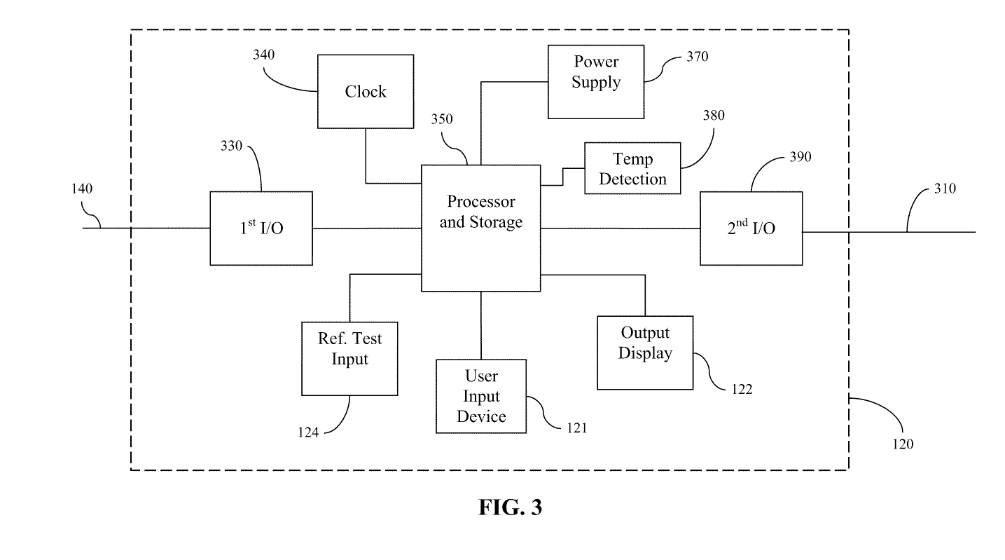

Referring to FIG. 3, receiver unit 120 may comprise first receiver I/O components 330 for communicating with transmitter device 110 (FIG. 1), second receiver I/O components 390 for communicating with external devices, receiver processor and storage 350, reference test input 124, output/display 122 and user input device 121. Receiver unit 120 may be further configured to transmit data by second receiver I/O components 390 over communication link 310 to a data processing terminal or other remote device for evaluating the data received by receiver unit 120.

In certain embodiments, the reference test interface 124 includes a glucose level testing portion to receive a manual insertion of a glucose test strip, and thereby determine and display the blood glucose level of the test strip on the output 122 of the receiver unit 120. This manual testing of glucose can be used to calibrate sensor 101 (FIG. 1). The user input device 121 of receiver unit 120 may be configured to allow the user to enter information into receiver unit 120 as needed. In one aspect, the user input device 121 may include one or more keys of a keypad, a touch-sensitive screen, or a voice-activated input command unit. The temperature detection section 380 is configured to provide temperature information of receiver unit 120 to the receiver processor 350, while the clock 340 provides, among others, real time information to the receiver processor 350. The receiver 120 and components, such as the processor 350, may be powered by power supply 370, which in some embodiments may include a battery.

In a further embodiment, receiver unit 120 may be configured to receive a blood glucose (BG) measurement over a communication link from, for example, a glucose meter, e.g., wirelessly or via a wired connection. In still a further embodiment, the user or subject using analyte monitoring system 100 may manually input a BG value using, for example, user input device 121.

Communication link 140 may include one or more of an RF communication protocol, an infrared communication protocol, a Bluetooth® enabled communication protocol, an 802.11x wireless communication protocol, a Zigbee® transmission protocol, or an equivalent wireless communication protocol which would allow secure, wireless communication of several units (for example, per HIPAA requirements) while avoiding potential data collision and interference.

Additional detailed description of the continuous analyte monitoring system, its various components including the functional descriptions of the transmitter are provided in U.S. Pat. No. 6,175,752 issued Jan. 16, 2001 entitled “Analyte Monitoring Device and Methods of Use”, and in U.S. Pat. No. 7,811,231 issued Oct. 12, 2010, entitled “Continuous Glucose Monitoring System and Methods of Use”, each assigned to the Assignee of the present application, and each of which are incorporated herein by reference for all purposes.

The following sections describe the components of transmitter device 110 in further detail. In one embodiment of the present disclosure, sensor 101 is physically positioned in or on the body of a user whose analyte level is being monitored. Sensor 101 may be configured to continuously sample the analyte level of the user and convert the sampled analyte level into a corresponding data signal for input into transmitter electronics 111. Alternatively, sensor 101 may be configured to sample analyte levels on-demand.

In general, sensors in accordance with the present disclosure operate electrochemically, through an arrangement of electrodes comprising sensor layers, e.g., by generating an electrical current proportional to the amount of analyte present. In some embodiments, such signal is related to the volume of a redox reaction of the analyte (and indicative of analyte concentration), catalyzed by an analyte-specific oxidizing enzyme. In some embodiments, ions, such as metallic ions, are provided as an electron transfer agent in the sensor system, and are kept by suitable mechanisms from diffusing away from the electrodes. Embodiments exist in which the number of electrodes provided to bring about and detect the level of these reactions is two, three or a greater number.

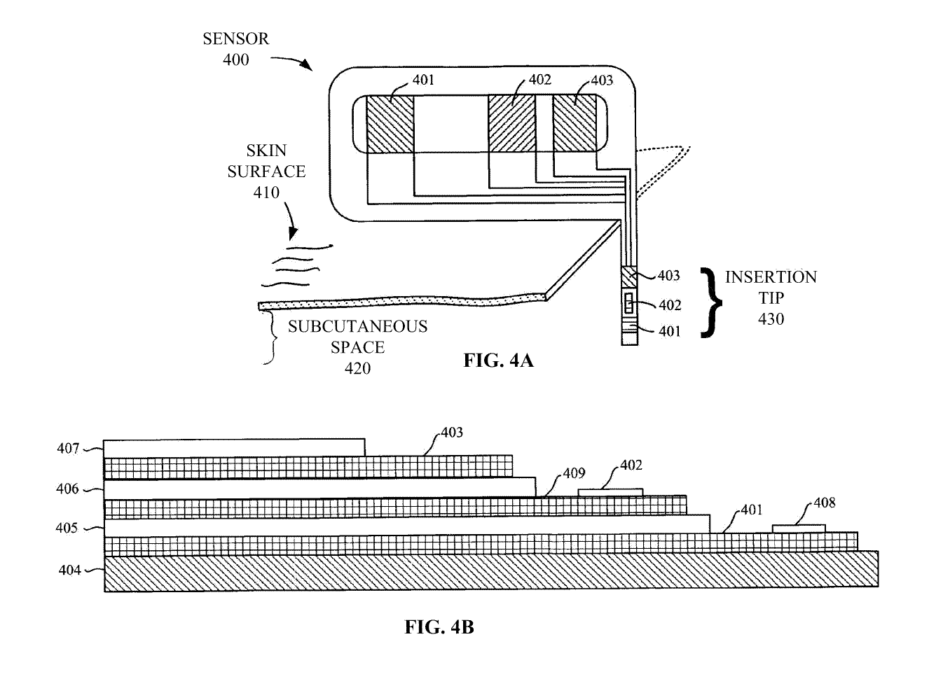

FIG. 4A shows a perspective view of an embodiment of an electrochemical analyte sensor 400 of the present disclosure having a first portion (which in this embodiment may be characterized as a major or body portion) positionable above a surface of the skin 410, and a second portion (which in this embodiment may be characterized as a minor or tail portion) that includes an insertion tip 430 positionable below the skin, e.g., penetrating through the skin and into, e.g., the subcutaneous space 420, in contact with the user's biofluid such as interstitial fluid. Contact portions of a working electrode 401, a reference electrode 402, and a counter electrode 403 are positioned on the portion of the sensor 400 situated above the skin surface 410. Working electrode 401, a reference electrode 402, and a counter electrode 403 are shown at the second section and particularly at the insertion tip 430. Traces may be provided from the electrode at the tip to the contact, as shown in FIG. 4A. It is to be understood that greater or fewer electrodes may be provided on a sensor. For example, a sensor may include more than one working electrode and/or the counter and reference electrodes may be a single counter/reference electrode, etc.

FIG. 4B shows a cross sectional view of a portion of the sensor 400 of FIG. 4A. The electrodes 401, 402 and 403 of the sensor 400 as well as the substrate and the dielectric layers are provided in a layered configuration or construction. For example, as shown in FIG. 4B, in one aspect, the sensor 400 (such as the sensor 101 FIG. 1), includes a substrate layer 404, and a first conducting layer 401 such as carbon, gold, etc., disposed on at least a portion of the substrate layer 404, and which may provide the working electrode. Also shown disposed on at least a portion of the first conducting layer 401 is a sensing component or layer 408, discussed in greater detail below. The area of the conducting layer covered by the sensing layer is herein referred to as the active area. A first insulation layer such as a first dielectric layer 405 is disposed or layered on at least a portion of the first conducting layer 401, and further, a second conducting layer 402 may be disposed or stacked on top of at least a portion of the first insulation layer (or dielectric layer) 405, and which may provide the reference electrode.

In one aspect, conducting layer 402 may include a layer of silver/silver chloride

(Ag/AgCl), gold, etc. A second insulation layer 406 such as a dielectric layer in one embodiment may be disposed or layered on at least a portion of the second conducting layer 409. Further, a third conducting layer 403 may provide the counter electrode 403. It may be disposed on at least a portion of the second insulation layer 406. Finally, a third insulation layer 407 may be disposed or layered on at least a portion of the third conducting layer 403. In this manner, the sensor 400 may be layered such that at least a portion of each of the conducting layers is separated by a respective insulation layer (for example, a dielectric layer). The embodiment of FIGS. 4A and 4B show the layers having different lengths. Some or all of the layers may have the same or different lengths and/or widths.

In addition to the electrodes, sensing layer and dielectric layers, sensor 400 may also include a temperature probe, a mass transport limiting layer, a biocompatible layer, and/or other optional components (none of which are illustrated). Each of these components enhances the functioning of and/or results from the sensor.

Substrate 404 may be formed using a variety of non-conducting materials, including, for example, polymeric or plastic materials and ceramic materials. (It is to be understood that substrate includes any dielectric material of a sensor, e.g., around and/or in between electrodes of a sensor such as a sensor in the form of a wire wherein the electrodes of the sensor are wires that are spaced-apart by a substrate).

Although the sensor substrate, in at least some embodiments, has uniform dimensions along the entire length of the sensor, in other embodiments, the substrate has a distal end or tail portion and a proximal end or body portion with different widths, respectively, as illustrated in FIG. 4A. In these embodiments, the distal end 430 of the sensor may have a relatively narrow width. For in vivo sensors which are implantable into the subcutaneous tissue or another portion of a patient's body, the narrow width of the distal end of the substrate may facilitate the implantation of the sensor. Often, the narrower the width of the sensor, the less pain the patient will feel during implantation of the sensor and afterwards.

For subcutaneously implantable sensors which are designed for continuous or semi-continuous monitoring of the analyte during normal activities of the patient, a tail portion or distal end of the sensor which is to be implanted into the patient may have a width of about 2 mm or less, e.g., about 1 mm or less, e.g., about 0.5 mm or less, e.g., about 0.25 mm or less, e.g., about 0.15 or less. However, wider or narrower sensors may be used. The proximal end of the sensor may have a width larger than the distal end to facilitate the connection between the electrode contacts and contacts on a control unit, or the width may be substantially the same as the distal portion.

Electrodes 401, 402 and 403 are formed using conductive traces disposed on the substrate 404. These conductive traces may be formed over a smooth surface of the substrate or within channels formed by, for example, embossing, indenting or otherwise creating a depression in the substrate. The conductive traces may extend most of the distance along a length of the sensor, as illustrated in FIG. 4A, although this is not necessary. For implantable sensors, particularly subcutaneously implantable sensors, the conductive traces typically may extend close to the tip of the sensor to minimize the amount of the sensor that must be implanted.

The conductive traces may be formed on the substrate by a variety of techniques, including, for example, photolithography, screen printing, or other impact or non-impact printing techniques. The conductive traces may also be formed by carbonizing conductive traces in an organic (e.g., polymeric or plastic) substrate using a laser. A description of some exemplary methods for forming the sensor is provided in U.S. patents and applications noted herein, including U.S. Pat. Nos. 5,262,035, 6,103,033, 6,175,752; and 6,284,478, the disclosures of which are herein incorporated by reference.

Certain embodiments include a Wired Enzyme™ sensing layer (such as used in the FreeStyle Navigator® continuous glucose monitoring system by Abbott Diabetes Care Inc.) that works at a gentle oxidizing potential, e.g., a potential of about +40 mV. This sensing layer uses an osmium (Os)-based mediator designed for low potential operation and is stably anchored in a polymeric layer. Accordingly, in certain embodiments the sensing element is redox active component that includes (1) Osmium-based mediator molecules attached by stable (bidente) ligands anchored to a polymeric backbone, and (2) glucose oxidase enzyme molecules. These two constituents are crosslinked together.

Examples of sensing layers that may be employed are described in U.S. patents and applications noted herein, including, e.g., in U.S. Pat. Nos. 5,262,035, 5,264,104, 5,543,326, 6,605,200, 6,605,201, 6,676,819, and 7,299,082, the disclosures of which are herein incorporated by reference.

Regardless of the particular components that make up a given sensing layer, a variety of different sensing layer configurations may be used. In certain embodiments, the sensing layer covers the entire working electrode surface, e.g., the entire width of the working electrode surface. In other embodiments, only a portion of the working electrode surface is covered by the sensing layer, e.g., only a portion of the width of the working electrode surface. Alternatively, the sensing layer may extend beyond the conductive material of the working electrode. In some cases, the sensing layer may also extend over other electrodes, e.g., over the counter electrode and/or reference electrode (or counter/reference is provided), and may cover all or only a portion thereof.

In some embodiments, the sensor is implantable into a subject's body for a period of time (e.g., three to seven days, or in some embodiments, longer periods of up to several weeks) to contact and monitor an analyte present in a biological fluid. In this regard, the sensor can be disposed in a subject at a variety of sites (e.g., abdomen, upper arm, thigh, etc.), including intramuscularly, transcutaneously, intravascularly, or in a body cavity. In one embodiment, the sensor can be a transcutaneous glucose sensor. Alternatively, the sensor can be a subcutaneous glucose sensor.

In some embodiments, sensor 400 is employed by insertion and/or implantation into a user's body for some usage period. In such embodiments, substrate 404 may be formed from a relatively flexible material to improve comfort for the user and reduce damage to the surrounding tissue of the insertion site, e.g., by reducing relative movement of the sensor with respect to the surrounding tissue.

While the embodiment illustrated in FIGS. 4A and 4B has three electrodes, other embodiments can include a fewer or greater number of electrodes. For example, a two electrode sensor can be utilized. The sensor may be externally-powered and allow a current to pass proportional to the amount of analyte present. Alternatively, the sensor itself may act as a current source in some embodiments. In some two-electrode embodiments, the sensor may be self-biasing and there may be no need for a reference electrode. An exemplary self-powered, two-electrode sensor is described in U.S. patent application Ser. No. 12/393,921, filed Feb. 26, 2009, entitled “Self-Powered Analyte Sensor,” which is hereby incorporated by reference in its entirety herein for all purposes. The level of current provided by a self-powered sensor may be low, for example, on the order of nanoamperes.

With continued reference to FIG. 2, electronics 111 are provided in transmitter device 110 to process signals from sensor 101 for transmission to receiver 120 through transmitter I/O 115.

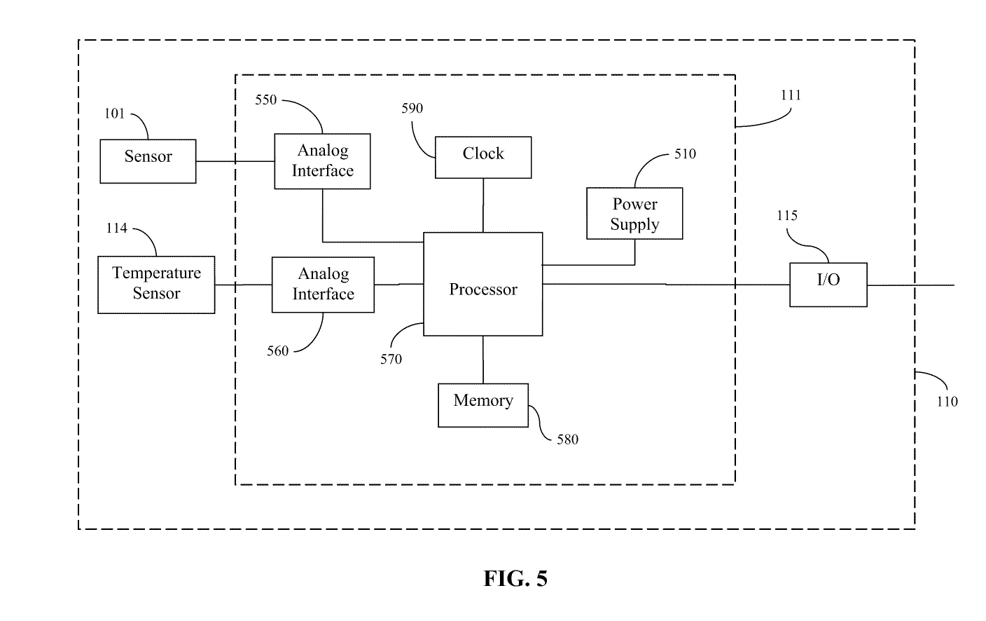

FIG. 5 schematically shows one embodiment of transmitter electronics 111 in further detail. An electrical current provided by sensor 101 is indicative of the concentration of analyte within the fluid exposed to the detecting areas of the sensor. In some embodiments, the current or voltage provided by sensor 101 may be initially processed with analog interface 550. A current measuring resistor (not shown) may be employed to develop a voltage proportional to sensor current. The analog interface may also, or alternatively, include, in some embodiments, an analog delay circuit (not shown), such as, but not limited to, an RC network, which can be used to provide memory of sequential measurements, averages of sequential measurements, or trend information.

Analog measurements will in general be further processed in some manner by transmitter electronics 111 for transmission through I/O section 115. In many embodiments, I/O section 115 will comprise a radio transmitter, although in others, I/O section 115 may present a plurality of direct electrical contact points to which a suitably adapted receiver unit 120 (FIG. 1) may be temporarily (or permanently) connected. In some embodiments, I/O section 115 can be driven directly from analog inputs without digital pre-conversion. In other embodiments, the analog data will be converted to digital data on board transmitter device 110, and processed in some manner in processor 570; in such embodiments, I/O 115 may include digital modulation functions (in the case, e.g., of RF I/O), or other digital interface (in the case of direct physical contact I/O).

In embodiments in which the sensor signal is digitally processed within transmitter device 110, analog interface 550 may further comprise one or more analog to digital converters (A/DCs) (not shown). In some embodiments, these may be “counting type” A/DCs that report, as an integer value, a time count to the change of state of a comparator, or some other count indicative of the analog input level. In embodiments in which analog circuits provide a plurality of analog measurements, such as, e.g., averaged, delayed or trending measurements, a separate A/DC may be used for each measurement, or, the measurements may be multiplexed and converted with a single A/DC.

Skin temperature may affect the sensitivity of sensor 101. Moreover, the ambient temperature around sensor 101 may affect the accuracy of the on-skin temperature measurement and ultimately the analyte value determined from the sensor signals. Temperature measurements may be used to adjust the analyte readings obtained from the analog interface 550. Transmitter device 110 may comprise one or more temperature sensors 114. In some embodiments, a temperature sensor, such as a thermistor (not shown), is provided in sensor 101, to get a skin temperature measurement proximate to the actual analyte measurement area. A second temperature sensor 114 (e.g., thermistor) may be provided in transmitter device 110 away from the on skin temperature sensor (for example, physically away from the temperature measurement section of the transmitter device 110), so as to provide compensation or correction of the on-skin temperature measurements due to the ambient temperature effects. In this manner, the accuracy of the estimated glucose value corresponding to the sensor signals may be improved. An analog interface 550 comprising, e.g., one or more A/DCs, may be provided for the temperature sensors. Digitized temperature information may be transmitted periodically, intermittently, or on-demand by the transmitter device 110 to the receiver unit 120, along with the sampled sensor signals.

Transmitter device 110 may further incorporate a leak detection circuit (not shown) coupled to the guard electrode (or other electrode) of sensor 101. The leak detection circuit may be configured to detect leakage current in the sensor 101 to determine whether the measured sensor data are corrupt or whether the measured data from the sensor 101 is accurate.

In some embodiments, processor 570 may have minimal functionality, or may be eliminated entirely in embodiments in which analog signals may be passed directly to I/O section 115. In other embodiments, processor 570 may be a digital processor. A digital form of processor 570 may be embodied in discrete logic, a Field Programmable Gate Array (FPGA) or an Application Specific Integrated Circuit (ASIC). Processor 570 may comprise a software-programmable processor, such as a general purpose microprocessor. Or, processor 570 may be a digital signal processor (DSP), either programmable via software with stored instructions, or embodied in logic, such as an FPGA or ASIC.

In digital implementations of transmitter device 110, processor 570 may require clock 590 for its own clocking In addition, in certain embodiments, as will be discussed, additional data acquisition components, such as shift registers, etc., may also require clocking In such embodiments, clock 590 may be provided. Clock 590 may be used to provide regular clock pulses. In some embodiments, clock 590 may also comprise a real time clock.

In some embodiments, depending on the implementation, processor 570 may need to store and/or load various values. Such values may include data such as the identification information for the transmitter device 110, as well as current and/or past analyte measurements and/or calculated values. In such cases, memory 580 may be provided. However, in some embodiments, a separate memory such as memory 580 may be avoided, or memory requirements may be satisfied by a small read-only memory.

In some embodiments, power supply 510 will be provided. Sensor 101 may, in some embodiments, operate as a current source. In such embodiments, transmitter 110 can function without a separate power supply and will be provided without power supply 510. In embodiments in which power supply 510 is provided, it may be a continuous, self-contained power supply such as a battery (which may be rechargeable), a power supply that receives power from an external source, such as an RF-coupled power supply powered by RF supplied during on-demand sessions by receiver unit 120, a power supply that receives power from a magnetic induction device on-board transmitter unit 110 that is activated by user movement, a solar-powered supply, and/or a capacitor or other storage device that is otherwise charged. See, e.g., U.S. patent application Ser. No. 12/807,278, filed Aug. 31, 2010, entitled “Medical Devices and Methods”, the disclosure of which is incorporated by reference herein for all purposes.

In some embodiments, transmitter processor 570 may operate in low power modes in the non-operating state, for example, drawing no more than approximately 1 μA of current. One of the final steps during the manufacturing process of the transmitter device 110 may be to place the transmitter device 110 in a non-operating state (e.g., post-manufacture sleep mode), to extend shelf life. Similarly, in an on-demand system, processor 570 could be configured to go into a low-power state, e.g., greatly reduced processor clock rate, between on-demand sessions. If the on-demand system is configured to log data between on-demand sessions, then power settings could be reduced between the times for data logging.

In an on-demand system, as an alternative to (or to supplement) a self-contained power supply, receiver 120 may provide requisite power to transmitter device 110 during on-demand data communications sessions. Such power could be provided, for example, by electrical contacts engageable when the two devices are drawn together, by RF induction, as used in passive RFID technology, or by other methods. Further description of power provision and data communication can be found in U.S. patent application Ser. No. 12/807,278, filed Aug. 31, 2010, entitled “Medical Devices and Methods”, the disclosure of which is incorporated by reference herein for all purposes.

In a CGM system, the transmitter device 110 may be configured to transmit the encoded sampled data signals at a fixed rate (e.g., at one minute intervals) after the completion of the initial power on procedure. Likewise, the receiver unit 120 may be configured to detect such transmitted encoded sampled data signals at predetermined time intervals. In some embodiments, the 433 MHz RF band, a band limited to short communications, is used for such purposes. In such embodiments, data is encoded in small packets, each comprising current and the immediately preceding sensor and/or temperature measurements, and potentially other data, such as sensitivity-related parameters for the specific transmitter.

Actual analyte concentrations will be expected to change over time, e.g., over several minutes or hours. However, signal artifacts and noise of various sorts can cause higher-frequency changes in the sensor signal. In some embodiments, for example, some CGM embodiments, transmitter device 110 may be configured to oversample the sensor signal, e.g., at a nominal rate of four samples per second, to allow an anti-aliasing filter in the transmitter device 110 to attenuate noise in the sensor measurements. For example, such a filter could be used to remove signal variations at frequencies above, e.g., 2 Hz, resulting (for example) from motion or movement of the sensor after placement or other inputs not related to analyte level.

Alternatively, an analog circuit might be used for noise reduction. In one embodiment, an RC network may be used for such purposes as described for example, in further detail in U.S. patent application Ser. No. 12/807,278, filed Aug. 31, 2010, entitled “Medical Devices and Methods”, the disclosure of which is incorporated by reference herein for all purposes. Noise reduction based on analog filtering may be combined with some averaging of digital data as well. This could be data sampled at a rate that would otherwise be used for logging, or at some faster rate for more selective noise reduction. Techniques for averaging collected data will be further discussed at a later point in this disclosure.

An RF transmitter incorporated in I/O 115 of transmitter device 110 may be configured for operation, for example, in the frequency band of 315 MHz to 322 MHz, 433 MHz, or 2.45 GHz, or at other RF frequencies. Further, in one embodiment, the RF transmitter may be configured to modulate the carrier frequency by performing Frequency Shift Keying (FSK) and Manchester encoding. In other embodiments, On-Off Keying (OOK) may be used. In some embodiment, the data transmission rates may be, e.g., 9,600, 14,400, 19,200, 28,800, or 38,400 symbols per second or other data rates, with a minimum transmission range for communication with the receiver unit 120.

In an on-demand system, receiver unit 120 will be configured to obtain data from transmitter device 110 on user command, such as when the user causes data transfer to be initiated by moving receiver unit 120 into physical proximity with transmitter device 110, and may include positive user actuation, e.g., pressing a button or other input. The distance of proximity will generally be a smaller distance than the normal distance separating the transmitter and receiver during the usual operation of a CGM system. Such proximity might be less than 12 inches, or less than five inches, or even less than one millimeter, such as, for example, in embodiments in which receiver 120 is temporarily docked with transmitter 110 through a mechanical interface, for on-demand data transmission.

In an on-demand embodiment, communication may be bi-directional: receiver unit 120 may send a signal to transmitter device 110 to begin an on-demand data transmission. The advance transmission by receiver unit 120 may also comprise an identification of a “clear channel” frequency for transmitter device 110 to send on. Such a protocol may be useful where, e.g., the 2.45 GHZ band (which, under regulations in many countries only permits transmission on a clear channel) is used in order to take advantage of higher bandwidth and the ability to send longer messages, particularly, e.g., where it is desired to send a sequence of measurements and/or average or trend data in a single transmission. Additional description of data communication can be found in U.S. patent application Ser. No. 12/807,278, filed Aug. 31, 2010, entitled “Medical Devices and Methods”, the disclosure of which is incorporated by reference herein for all purposes.

Transmitter device 110 may be further configured to detect one or more states that may indicate when a sensor is inserted, when a sensor is removed from the user, and further, may additionally be configured to perform related data quality checks so as to determine when a new sensor has been inserted or transcutaneously positioned under the skin layer of the user and has settled in the inserted state such that the data transmitted from the transmitter device 110 does not compromise the integrity of signal processing performed by the receiver unit 120 due to, for example, signal transients resulting from the sensor insertion. Based on power consumption or complexity constraints, these functions may also be omitted from transmitter device 110.

Transmitter device 110 may provide “passive” notification of functions that will notify the user of the patient of an issue that has developed, for example when an ongoing or predetermined routine has malfunctioned or raised an alarm, but which will not interrupt processing. The ongoing routine or the predetermined routine being executed may include, e.g., one or more of performing a finger stick blood glucose test (for example, for purposes of periodically calibrating the sensor unit 101), or any other processes that interface with the user interface, for example, on the receiver/monitor unit 120 (FIG. 1) including, but not limited to the configuration of device settings, review of historical data such as glucose data, alarms, events, entries in the data log, visual displays of data including graphs, lists, and plots, data communication management including RF communication administration, data transfer to the data processing terminal, or viewing one or more alarm conditions with a different priority in a preprogrammed or determined alarm or notification hierarchy structure. These functions may also be omitted from transmitter device 110, based on power consumption or complexity constraints.

In this manner, in certain embodiments, the detection of one or more alarm conditions may be issued or notified to the user or the patient, without interrupting or disrupting an ongoing routine or process in, for example, the receiver 120 of the data monitoring and management system. Accordingly, it should be appreciated that there are a broad range of options for implementing electronics 111 within transmitter device 110, and that the choice of sensors, the manner and mode of communications between transmitter device 110 and receiver unit 120, among other factors, may influence the choice and interconnection of components within electronics 111.

In another embodiment, the receiver 120 can issue or notify the user of an anticipated or projected physiological condition, such as for example, an elevated analyte level, a depressed analyte level, or an analyte trend that is developing over time. For example, as described herein, the on-demand analyte monitoring system differs from a continuous glucose monitor in that the on-demand only provides analyte data information when queried or commanded by the user. As such, the projected alarm for a physiological condition for an on-demand system would necessarily differ from a typical continuous analyte monitor, such as CGM systems, which sends analyte data to the receiver 120 continuously or periodically, e.g., every 30 seconds, because the data is transmitted to the receiver 120 only when commanded or otherwise prompted or requested. Accordingly, as described in this disclosure, the on-demand system may be configured to provide the user projected alarm notification when the user queries or commands analyte level data by close proximity and/or other positive user actuation techniques described here.

The data processing to determine a projected alarm condition can be the same for an on-demand system as for a CGM systems. A processing method can be used to determine a line based on the best linear fit of the most recent 15 minutes of glucose data, and determine a projected glucose value based on an extension of this line from the most recent glucose data point for a predetermined time period, such as 10, 20 or 30 minutes.

In some instances, an on-demand projected alarm and a continuous monitor projected alarm can be different. In one example, the alarm employed with a continuous monitor is automatic. In other words, the receiver is configured to enunciate or issue an alarm when the receiver retrieves a new data point, processes it along with some number of past data and determines that the projected alarm condition has been met. In contrast, the on-demand projected alarm is a notification associated with the glucose result presented to the patient upon their command. For example, for an on-demand projected alarm, when the receiver 120 retrieves glucose data from the transmitter 110 in response to a user-initiated command, it then displays the glucose value and perhaps a glucose trend indicator. In addition, the processor of receiver 120 performs the projected alarm processing and if a projected alarm condition is met, then the display would include an indication of the projected alarm. Non-limiting examples of this indication may include a special icon, a text string such as “projected low detected”, a flashing LED, or an audio enunciation. The display indication may also include the time when, based on a linear projection, the glucose is expected to cross the alarm threshold. For a glucose analyte monitor, the on-demand projected alarm may be used to detect potential future low glucose or high glucose conditions. This projected scheme may be used, however, with any on-demand or continuous analyte measurement system where it is important to determine future analyte level.

In certain embodiments, data analysis, including analyte level, rate of change, and trend analysis, may all be performed by processor 350 (FIG. 3) of receiver 120. In such embodiments, transmitter 110 (FIG. 1) may be configured for data transmission of signals received from sensor 101. The signals from sensor 101 are transmitted by transmitter 110 to receiver 120 via communication link 140, as described above. In other embodiments, transmitter 110 may include a memory 580 (FIG. 5) for storing previously monitored analyte related signals received from sensor 101, such as analyte related signals received from sensor 101 over a predetermined time period such as, for example, the prior 15 minutes, 30 minutes, 45 minutes, and so on. Processor 570 of transmitter 110 may be configured for communicating with sensor 101 and temperature sensor 114 and for controlling data transmission with receiver 120, but may be not configured for performing data analysis of signal data received from sensor 101. By limiting the analysis operations performed by processor 570 of transmitter 110, power usage of transmitter 110 may be minimized and accordingly the battery life of the transmitter 110 may be extended.

In the embodiments where data analysis is performed by the receiver 120, processor 350 (FIG. 3) of receiver 120 is configured to analyze the signals received from the sensor electronics or transmitter 110 in signal communication with sensor 101. As described above, in an on-demand configuration, receiver 120 may receive data from transmitter 110 only on an on-demand basis, upon request from the user by actuating a button or a switch on receiver 120 to transmit a request command. In certain embodiments, the transmitter 110 transmits the current signal data from sensor 101 along with a stored data retrieved from memory 580 that span a predetermined time period, such as the preceding 15 minutes of data from sensor 101. In certain embodiments, the amount of preceding time of sensor data may be programmable. For example, a user may select whether to receive a preceding 15 minutes of sensor data for analysis, or a data spanning a different time period, such as 10 minutes of stored data, 20 minutes, 30 minutes, 45 minutes, 1 hour, or more. In other embodiments, the amount of time of stored data received may be based on a profile determined or programmed by a medical professional, which may be based on, among others, a specific user's physiological condition, such as a user's response time to an insulin injection or a user's response time or ingestion time to carbohydrate intake. In other embodiments, the profile may be determined by the receiver 120, by analyzing the previously stored data. Further, the determined amount of time of data to receive may be based on a time of day schedule, such that varying time lengths may be used based on a time of day corresponding to a normal meal time, exercise time, or sleep time, or a time since the last on-demand command for receiving data was initiated.

Upon receipt of signals including multiple data points from the transmitter 110 in response to the data request or command transmitted from the receiver, receiver processor 350 may perform analysis on the received data. Such analysis may include a determination of the current analyte level, a determination of a current status of an anticipated physiological condition, a projected alarm determination, a rate of change of the monitored analyte level determination, a trend analysis, or other functions such as a calibration or command to display the analyzed data on display 122 of receiver 120. For example, based on sensor data for a current analyte level as determined by sensor 101 along with sensor data for the preceding 15 minutes, which may include a series of time spaced sensor measurement, may be analyzed by receiver processor 350 to determine a current trend or direction of the analyte level movement or rate of change of analyte level of the user.

In certain embodiment, having the current real time analyte level information in addition to the current status or direction of the analyte level fluctuation, provide clinically advantageous function to notify the user who has requested, via activation or transmission of the command for the current glucose level, in response to such request, to also be provided with the projected analyte level information. For example, visually, on display 122 of receiver 120, the current level may be represented as a dot or an indicator on the display 122, and in addition, an overlayed directional arrow whose direction and angle provide visual indication of the direction the analyte level monitored is moving towards, as well as the rate at which the monitored analyte level is moving towards the determined level.

In certain embodiments, the rate of change determination may be used to calculate the current trend of analyte level. Such a trend analysis may allow processor 350 to estimate a predicted future analyte level, which may be displayed or otherwise output to the user. In certain embodiments, the trend analysis and received sensor data may be compared with previously received sensor data, which may be stored in a memory 580 of receiver 120. Comparison of the current and immediately preceding sensor data with past stored sensor data may allow processor 350 of receiver 120 to establish a user's analyte, such as glucose, profile. The analyte profile may be applied to future received data sets to better estimate a future analyte concentration level in conjunction with rate of change and current analyte level information.

In the manner described, in certain embodiments, when a user transmits a request for glucose level information, in response to such request, the user is automatically provided with the current glucose level information in addition to the determined direction of fluctuation information, graphically, numerically or otherwise provided to the user to easily and readily determine a current snap shot of the glucose level as well as the manner or direction in which the glucose level is fluctuating.

An additional aspect of the above invention is to incorporate a continuous projected (or simple threshold) alarm in the transmitter device 110 electronics, even though the receiver 120 operates as an on-demand device. The transmitter device 110 processor may perform the alarm detection processing. The transmitter device 110 may also include some simple form of alarm enunciator such as an audio or vibratory device, or a LED. When the alarm occurs on the transmitter device 110, then the user may retrieve the on-demand data using the receiver 120 and the receiver display would indicate the alarm.

In one embodiment, the transmitter electronics, described above, can transmit current analyte concentration level data to the receiver 120, as well as, past analyte or historic analyte data concentrations, e.g., data of analyte concentrations for the previous 15 minutes up to several hours. In this manner, the receiver 120 can be configured to process the data provided to it from the transmitter device to determine trend information. The trend information can be determined using many well understood methods. One method would be to perform a least squares fit to determine the slope of a best fit line. The slope is in units of glucose change per unit time. Another simple method would be to determine the slope as:

(Present/Current analyte concentration)−(analyte concentration 15 minutes ago)/(15 minutes)

The trend information determined by the on-demand system can be utilized by the receiver 120 to perform projected alarm calculations, using for example, the following relationship:

(Projected analyte concentration)=(Present/current analyte concentration)+(slope of line)*(predetermined time).

If the projected analyte concentration exceeds an alarm threshold, then the alarm condition is considered satisfied, where the predetermined time in the above relationship may be 10, 20 or 30 minutes, for example.

The calculation, in some embodiments, can be prompted by the user requesting an analyte data reading from the monitoring system on-demand. As distinct from the continuous monitor, where the projected alarm processing module has inputs from data logged periodically as received automatically from the transmitter, the on-demand system inputs glucose data to the projected alarm processing module as retrieved by a single user initiated command.

Based on the current analyte data demanded by the user and the past analyte data, e.g., historic data stored in the memory of the receiver 120, and/or trend information, a physiological alarm condition can be detected or anticipated. The receiver, as described below, then is configured to issue alarm notification to the user on-demand. The alarm condition can take the forms of a variety of modes. For example, the alarm condition may include a visual notification, such as a pop-up screen or icon displayed on the receiver user interface, an auditory alarm, such as a beep, siren, music, voiceover, or a tactile alarm such as vibration, or a combination thereof. In some embodiments, the alarm can be enabled and/or disabled by the user, as described in U.S. patent application Ser. No. 12/761,387 filed Apr. 15, 2010, the disclosure of which is incorporated herein by reference for all purposes.

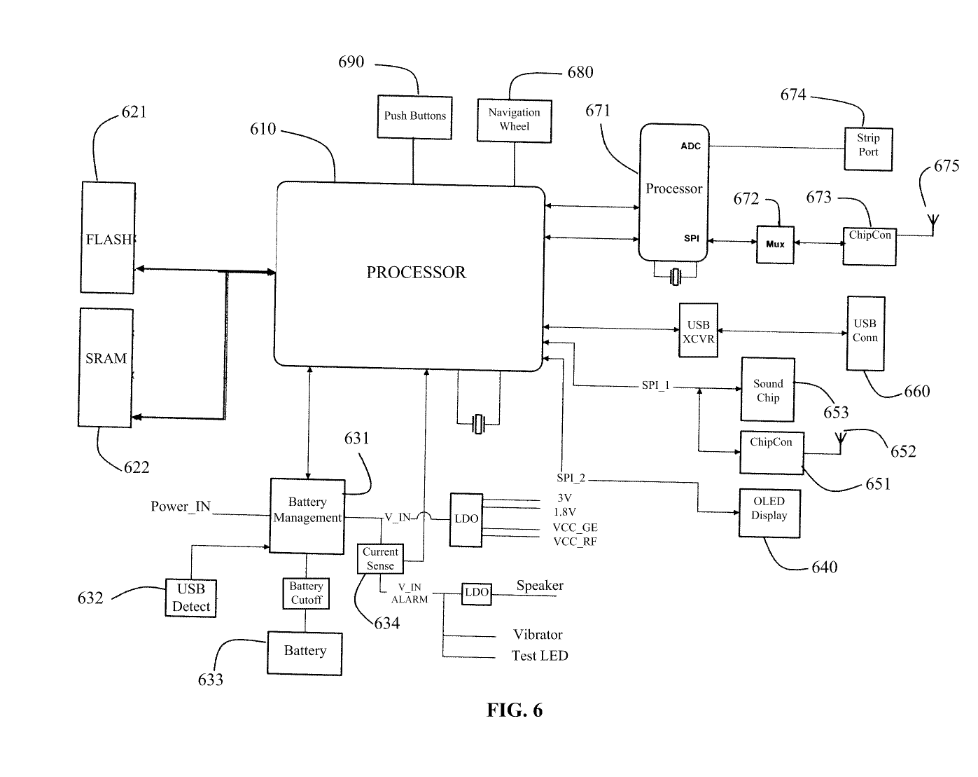

A block diagram of the receiver 120 in accordance with another exemplary embodiment is illustrated in FIG. 6. The receiver 120 may include one, two or more processors. In some embodiments, a first processor 610 with external SRAM 622 and Flash 621 is used. Also provided on the receiver 120 is an on-board transceiver IC 673 and antenna 675 which allow bi-directional RF communications. In some embodiments, an additional transceiver IC 651 and antenna 652 is provided for communication to an additional component, such as an insulin pump. In some embodiments, a sound synthesizer IC 653 is also provided.

A second processor 671 can be provided to handle analyte data acquisition, and receive data from the analyte strip port 674. The processor 610 also handles the interface for the RF communications with the sensor transceiver (673). It is understood that the receiver 120 may operate with one processor, or with a plurality of processors.

The receiver 120 can further include a user interface functionality which can be operated at least in part by processor 610 (also referred to interchangeably herein as “UI processor 610”). UI processor 610 can be responsive to the user interface through user controls, such as buttons 690 on the front and side of the case and the jog wheel or thumbwheel 680. UI processor 610 can also receive messages from the processor 671, e.g., when a glucose test strip is inserted to port 674, and from a USB interface 660. In some embodiments, control functionality of processor 671 includes interaction with the processor 671, updating the display 640, processing the received glucose data, maintaining a log of historical information, operating the sound synthesizer 653 and vibrator, interface with the radio 651. UI processor 610 also interfaces with the power supply 633 and power management circuitry 631.

Processor 671 communicates with other components through an asynchronous serial interface in some embodiments. In some embodiments, processor 671 is responsible for the following exemplary functions: Real-Time Clock, some power (battery) management functions, continuous glucose data processing, discrete glucose data processing, internal temperature monitoring, UI “watchdog” and reset functions, and RF Protocols for 433 MHz radio.

The receiver 120 can contain a power supply 633 which supplies power to the system. In some embodiments, when the receiver 120 is docked on a cradle with an AC adapter or plugged into a PC, power is provided via a pin of the USB connector. The battery charger and power path IC 631 will then route power to both the receiver electronics and the battery for recharging.

The receiver 120 can have several indicator elements for communicating with user, for example, to provide alarm output modes to issue alarm notifications. In some embodiments, a sound synthesizer IC 653, a coin cell vibrator, and a strip measurement LED are provided. The sound synthesizer 653 can play MIDI or wave sound files. Thus, various types of output modes can be associated with a particular alarm, as described below. The user can also select the vibrator as a silent alarm notification.

Two different radio ICs can be used by the receiver 120 for external communication. In some embodiments, transceiver 673 can be used for 433 MHz communication to the transmitter device 110, and transceiver 651 can be used for 2.4 GHz communication to a pump (not shown). Both radios can use an integrated on board antenna.

The receiver 120 can comprise a user interface (“UI”) incorporating the following components: the UI processor 610, a display 640, e.g., a full color organic light emitting diode (OLED) display, RAM 622, FLASH Memory 621, a USB connection, Sound synthesizer IC 660 and a speaker. It is understood that the UI may include additional or fewer components. For example, in some embodiments, a sound synthesizer and speakers may be omitted for a purely visual UI. In some embodiments, a display is omitted and user interface outputs are provided by sound and vibration.

In some embodiments, the UI includes a plurality of display screens which can be organized in an order, or hierarchy. Such arrangement allows the user to logically select operations of the receiver. Many of these operating screens can be provided with additional features, such as color, which allow the user to quickly recognize the severity or criticality of information (e.g., system information or critical blood glucose levels), or to recognize which system or aspect of the device is being affected (e.g., sensor, calibration, etc.). The UI described herein can also be suitable for use with our medical systems, such as, e.g., insulin infusion pumps, EKG devices, etc.

In the description that follows, the following terms are used in connection with the user interface: “Screentype” refers to the template for layout and function of a screen, whose components are specified for each specific instance of a screen. “Softkey” refers to the left and right buttons on the face of the receiver 120. “Softkey Label” refers to the text in the bottom line of the screen placed above either softkey to indicate the current function of the softkey press. “Scroll Wheel” or “Jog Wheel” refers to the physical scroll-action control on the side of the receiver 120 that has inputs of up, down (also scroll up or scroll down) and select (e.g., push in). Such functions could also be carried out by a pair of “up” and “down” buttons and a “select” button. The “back button” refers to a button on the side of the receiver 120. (See, e.g., FIG. 1.)

It is understood that the UI is suitable for use with a greater or fewer number of buttons. The “scroll indicator” refers to the sidebar displayed on the screen to assist in navigation through a list of selectable items. The “title” refers to the header text displayed on screen to differentiate screens from one another. “Wrap Navigation” refers to the feature that allows the cursor to loop back to the top of a list when cursor reaches the last item of a list and the user continues to select “down” direction. This function is also applicable to the “up” direction and looping to the bottom of list. The “Cursor” refers to the visual indicator of the area of action for the scroll wheel or other user inputs. “Highlight” refers to an area of solid color.

The UI can be configured to include a number of screentypes which provide different functionality and information to a user. In some embodiments, the receiver and more particularly the user interface includes screentypes, such as, e.g., home screen, menu screen, message screen, editable screen, display day/date screen, editable event, blood glucose (BG) history screen, event history screen, line graph screen, animation screen, BG result screen.

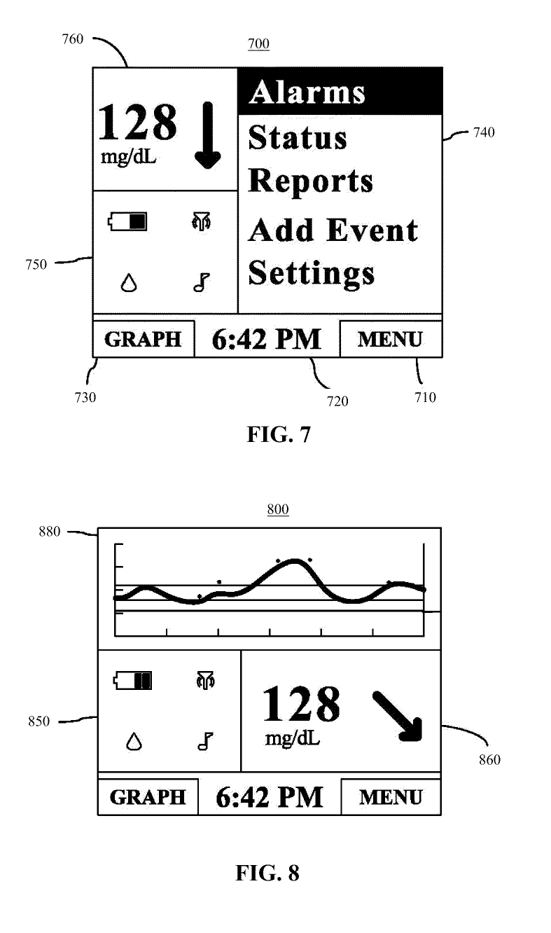

The home screentype may be configured by the user to several configurations, such as an activity mode and an information mode. An activity mode homescreen 700 is illustrated in FIG. 7. The home screen can be designed to present information related to user state and system state, and provides options to initiate activities. User state information may include glucose value, rate of change, insulin, meals and other user-related activities, which may all be presented in numbers, text, icons or graphically. System state information may include information related to sensor status, calibration status, radio status, battery status, and alarm status. This information may be presented in text, numbers, icons or graphically. In addition to communicating user and system status, the home screen may include user selectable options which link to functions included in the user interface. In one embodiment of the home screen, the home screen includes softkey labels 730/710, and the time is displayed 720. The home screen may display one or more information panels. For example, the home screen may include a cursor-selectable menu 740 in an information panel. In some embodiments, panel 760 can include a glucose information screen, which displays glucose data and trend information.

In some embodiments, another panel 750 may include icons such as battery level/battery charge in percentage of charge, audio selections (vibration/volume/muting), transmitter radio status, blood drop required for calibration, or an hourglass icon indicating the user must wait to certain functions to be completed before proceeding. In some embodiments, the home screen may include the menu items such as: Sensor, Alarms, Status, Reports, Add Event, and Settings. Additional menu items may include CGM status, Graph, and Main. If the system is used an insulin infusion system, menu items may be provided relating to the dispensing of an insulin bolus, etc.

One example of an information mode homescreen 800 is illustrated in FIG. 8. Homescreen 800 can be provided with a screen 880 in which historical analyte data is indicated, for example, by plotting. Event data, such as discrete blood glucose measurements, insulin dosing, or meal times may be plotted on the graph as distinct points or otherwise indicated. In some embodiments, one panel 860 can provide a glucose information screen, which can display glucose data, such as continuous glucose information, and trend information. In some embodiments, panel 850 may include icons such as battery level/battery charge in percentage of charge, audio selections (vibration/volume/muting), transmitter radio status, blood drop required for calibration, or an hourglass icon indicating the user must wait to certain functions to be completed before proceeding.

The menu screentype includes a title, e.g., “Sensor.” In some embodiments, the menu screentype items are navigable using the scroll wheel and may (or may not) allow wrap navigation. The menu screentype has a scroll indicator when there is a list of more items than can be displayed at once on a single screen, e.g., greater than five. The menu screentype advances to a unique screen for each menu item selection. In some embodiments, the menu screentype allows menu item selection by pressing the scroll wheel. The menu screentype has a left and a right softkey label and softkey destination. The menu item text may be context sensitive, e.g., in different states the text may be different.

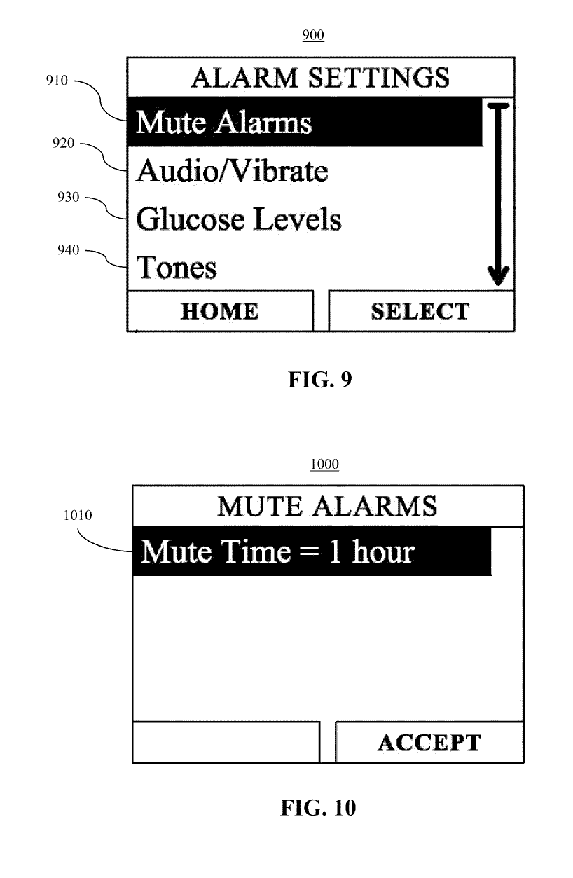

In one embodiment, the “Alarm” menu screentype 900 may include menu screen items, e.g., “Mute Alarms” 910, “Audio/Vibrate” 920, “Glucose Levels” 930, and “Tones” 940, etc., as shown in FIG. 9. With respect to the “Mute Alarms” menu, the user is presented with different options if the alarms are not muted or are already muted. For example, to mute the alarm, the user is presented in screen 1000 with the duration of muting 1010 (which depends on the level of the alarm or alert) and the option to accept muting, and subsequently to confirm muting (FIG. 10). If the user wishes to un-mute the alarm, the user is presented with the option of confirming un-muting. Upon selecting the menu item “Audio/Vibrate” the user is provided with the option of selecting an audio level (high, medium, low, silent). The UI provides the user with the option to change from audible to vibratory to silent alarm presentations with a single step. The UI provides the user with the option to configure alarm presentation, e.g., by providing unique tones, or vibrate. The UI also allows the user to select an option in which a vibratory alert trumps an audible setting for individual alarms. For example, vibrate alerts may be presented for a specific alarm type despite the global audible setting because vibrate setting is more discreet.

Certain embodiments of the present disclosure include an analyte monitoring system that may comprise an analyte sensor adapted to measure one or more analyte concentrations present in a bodily fluid of a user and to generate signals corresponding an analyte concentration, sensor electronics in signal communication with the analyte sensor and configured to transmit the signals corresponding to the measured analyte concentration in response to a command, and a receiver configured to generate and transmit the command to the sensor electronics, and in response to the transmitted command, to receive the signals corresponding to the measured analyte concentration from the sensor electronics, the received signals including multiple data points corresponding to the measured analyte concentration over a predetermined time period, the receiver including a display configured to output the received one or more signals from the sensor electronics, the receiver configured to determine a real time analyte concentration level and a current status of an anticipated physiological condition based on the received multiple data points, and further wherein the receiver is configured to output a notification associated with the determined current status of the anticipated physiological condition, wherein the sensor electronics transmits the multiple data points corresponding to the measured analyte concentration over the predetermined time period in a single data transmission to the receiver in response to the received command, and further wherein the receiver outputs an indication of the determined real time analyte concentration level and an indication of the determined current status of the anticipated physiological condition on the display.

In certain aspects, the receiver may be configured to determine the current status of the anticipated physiological condition by determining a rate of change of the monitored analyte level based on the received multiple data points corresponding to the measured analyte concentration over the predetermined time period.

In certain aspects, the receiver may be configured to determine the current status of the anticipated physiological condition by performing a statistical analysis of the monitored analyte level based on the received multiple data points corresponding to the measured analyte concentration over the predetermined time period.

In certain aspects, the current status of the anticipated physiological condition may include a projected alarm.

In certain aspects, the receiver may concurrently output the indication of the determined real time analyte concentration level and the indication of the determined current status of the anticipated physiological condition on the display.

In certain aspects, the indication of the determined real time analyte concentration level and the indication of the determined current status of the anticipated physiological condition may be displayed simultaneously and overlapping at least a portion of the display area.

In certain aspects, the receiver may be configured to determine trend information based on the multiple data points received from sensor electronics.

In further aspects, the receiver may be adapted to determine an anticipated analyte concentration level based on the trend information and/or the rate of change of analyte concentration.

In yet further aspects, the receiver may be programmed to issue a notification based on the anticipated analyte concentration level.

In certain aspects, the predetermined time period may be programmable by the user.

In certain aspects, the receiver may be configured to determine a glucose profile information based on the multiple data points received from the sensor electronics in response to the command, and based on data points received in prior communication from the sensor electronics and retrieved from a storage unit of the receiver.

In certain aspects, the receiver may be programmed to determine an anticipated analyte concentration level based on the glucose profile information.

In certain aspects, the notification may be a visual indicator displayed by the display of the receiver.

In a further aspect, the visual indicator may be a popup screen or an icon.

In certain aspects, the notification may be an audio indicator or a tactile indicator.

In certain aspects, the tactile indicator may comprise a vibrating component in the receiver.

In certain aspects, the anticipated physiological condition may be an elevated analyte concentration.

In further aspects, the elevated analyte concentration may include a hyperglycemic condition.

In certain aspects, the anticipated physiological condition may be a depressed analyte concentration.

In further aspects, the depressed analyte concentration may include a hypoglycemic condition.

In certain aspects, the receiver may be configured to permit the user to enable or disable the notification.

In certain aspects, the receiver may include a transceiver configured for bidirectional communication with the sensor electronics initiated by the transmitted command.

In further aspects, the transceiver may be configured to communicate via a radiofrequency link.

In yet further aspects, the transceiver may be configured to communicate via radiofrequency identification.

In certain aspects, the command may comprise positioning the receiver within a predetermined distance from sensor electronics.

In certain aspects, the received signals may include multiple data points corresponding to the measured analyte concentration over the predetermined time period includes substantially equally time spaced data points over the predetermined time period.

In certain aspects, the predetermined time period may include one of one hour, 45 minutes, 30 minutes, 20 minutes, 15 minutes, or 10 minutes.

Certain embodiments of the present disclosure may include a method comprising transmitting a command to a sensor electronics, wherein the command includes a request for analyte sensor data, receiving the analyte sensor data from the sensor electronics, the analyte sensor data corresponding to a measured analyte concentration present in a bodily fluid of a user, wherein the received analyte sensor data includes multiple data points corresponding to the measured analyte concentration over a predetermined time period, displaying the received analyte sensor data from the sensor electronics, determining a real time analyte concentration level and a current status of an anticipated physiological condition based on the received multiple data points, displaying an indication of the determined real time analyte concentration level and an indication of the determined current status of the anticipated physiological condition, and outputting a notification associated with the determined status of the anticipated physiological condition, wherein the analyte sensor data including the multiple data points corresponding to the measured analyte concentration over the predetermined time period is received in a single data transmission from the sensor electronics in response to the transmitted command determining the current status of the anticipated physiological condition by determining a rate of change of the monitored analyte level based on the received multiple data points corresponding to the measured analyte concentration over the predetermined time period.

Certain aspects may include determining the current status of the anticipated physiological condition by performing a statistical analysis of the monitored analyte level based on the received multiple data points corresponding to the measured analyte concentration over the predetermined time period.

In certain aspects, the current status of the anticipated physiological condition may include a projected alarm.

Certain aspects may include concurrently outputting the indication of the determined real time analyte concentration level and the indication of the determined current status of the anticipated physiological condition on the display.

In certain aspects, the indication of the determined real time analyte concentration level and the indication of the determined current status of the anticipated physiological condition may be displayed simultaneously.

Certain aspects may include determining trend information based on the multiple data points received from sensor electronics.

Certain aspects may include determining an anticipated analyte concentration level based on the trend information and/or the rate of change of analyte concentration.

Certain aspects may include issuing a notification based on the anticipated analyte concentration level.

In certain aspects the predetermined time period is programmable by the user.

Certain aspects may include determining a glucose profile information based on the multiple data points received from the sensor electronics in response to the command, and based on data points received in prior communication from the sensor electronics and retrieved from a storage unit.

Certain aspects may include determining an anticipated analyte concentration level based on the glucose profile information.

In certain aspects, the notification is a visual indicator.

In certain aspects, the visual indicator is a popup screen or an icon.

In certain aspects, the notification is an audio indicator or a tactile indicator.

In certain aspects, the anticipated physiological condition is an elevated analyte concentration.

In certain aspects, the elevated analyte concentration includes a hyperglycemic condition.

In certain aspects, the anticipated physiological condition is a depressed analyte concentration.

In certain aspects, the depressed analyte concentration includes a hypoglycemic condition.

Certain aspects may include permitting the user to enable or disable the notification.

In certain aspects, transmitting to and receiving from the sensor electronics includes transmitting and receiving via a radiofrequency link.

In certain aspects, the radiofrequency link includes radiofrequency identification.

In certain aspects, the received signals including multiple data points corresponding to the measured analyte concentration over the predetermined time period includes substantially equally time spaced data points over the predetermined time period.

In certain aspects, the predetermined time period includes one of one hour, 45 minutes, 30 minutes, 20 minutes, 15 minutes, or 10 minutes.

In certain embodiments, a device for processing analyte sensor data may comprise a processor, a transmitter operatively coupled to the processor and configured to transmit a command to a sensor electronics, wherein the command includes a request for analyte sensor data, a receiver operatively coupled to the processor and configured to receive the analyte sensor data from the sensor electronics, the analyte sensor data corresponding to a measured analyte concentration present in a bodily fluid of a user, wherein the received analyte sensor data includes multiple data points corresponding to the measured analyte concentration over a predetermined time period, a display operatively coupled to the processor and configured to display the received analyte sensor data from the sensor electronics, and a memory including instructions which, when executed by the processor, causes the processor to determine a real time analyte concentration level and a current status of an anticipated physiological condition based on the received multiple data points, display an indication of the determined real time analyte concentration level and an indication of the determined current status of the anticipated physiological condition on the display, and output a notification associated with the anticipated physiological condition, wherein the analyte sensor data including the multiple data points corresponding to the measured analyte concentration over the predetermined time period is received in a single data transmission from the sensor electronics in response to the transmitted command.

Various other modifications and alterations in the structure and method of operation of this disclosure will be apparent to those skilled in the art without departing from the scope and spirit of the embodiments of the present disclosure. Although the present disclosure has been described in connection with particular embodiments, it should be understood that the present disclosure as claimed should not be unduly limited to such particular embodiments. It is intended that the following claims define the scope of the present disclosure and that structures and methods within the scope of these claims and their equivalents be covered thereby.

What is claimed is:

1. An analyte monitoring system, comprising:
an analyte sensor adapted to measure one or more analyte concentrations present in a bodily fluid of a user and to generate signals corresponding an analyte concentration;
sensor electronics in signal communication with the analyte sensor and configured to transmit the signals corresponding to the measured analyte concentration in response to a command; and
a receiver configured to generate and transmit the command to the sensor electronics, and in response to the transmitted command, to receive the signals corresponding to the measured analyte concentration from the sensor electronics, the received signals including multiple data points corresponding to the measured analyte concentration over a predetermined time period, the receiver including a display configured to output the received one or more signals from the sensor electronics, the receiver configured to determine a real time analyte concentration level and a current status of an anticipated physiological condition based on the received multiple data points, and further wherein the receiver is configured to output a notification associated with the determined current status of the anticipated physiological condition;
wherein the sensor electronics transmits the multiple data points corresponding to the measured analyte concentration over the predetermined time period in a single data transmission to the receiver in response to the received command; and further
wherein the receiver outputs an indication of the determined real time analyte concentration level and an indication of the determined current status of the anticipated physiological condition on the display.

2. The analyte monitoring system of claim 1 wherein the receiver is configured to determine the current status of the anticipated physiological condition by determining a rate of change of the monitored analyte level based on the received multiple data points corresponding to the measured analyte concentration over the predetermined time period.

3. The analyte monitoring system of claim 1 wherein receiver is configured to determine the current status of the anticipated physiological condition by performing a statistical analysis of the monitored analyte level based on the received multiple data points corresponding to the measured analyte concentration over the predetermined time period.

4. The analyte monitoring system of claim 1 wherein the current status of the anticipated physiological condition includes a projected alarm.

5. The analyte monitoring system of claim 1 wherein the receiver concurrently outputs the indication of the determined real time analyte concentration level and the indication of the determined current status of the anticipated physiological condition on the display.

6. The analyte monitoring system of claim 5 wherein the indication of the determined real time analyte concentration level and the indication of the determined current status of the anticipated physiological condition are displayed simultaneously and overlapping at least a portion of the display area.

7. The analyte monitoring system of claim 1, wherein the receiver is configured to determine trend information based on the multiple data points received from sensor electronics.

8. The analyte monitoring system of claim 7, wherein the receiver is adapted to determine an anticipated analyte concentration level based on the trend information and/or the rate of change of analyte concentration.

9. The analyte monitoring system of claim 8, wherein the receiver is programmed to issue a notification based on the anticipated analyte concentration level.

10. The analyte monitoring system of claim 1, wherein the predetermined time period is programmable by the user.

11. The analyte monitoring system of claim 10, wherein the receiver is configured to determine a glucose profile information based on the multiple data points received from the sensor electronics in response to the command, and based on data points received in prior communication from the sensor electronics and retrieved from a storage unit of the receiver.

12. The analyte monitoring system of claim 11, wherein the receiver is programmed to determine an anticipated analyte concentration level based on the glucose profile information.

13. The analyte monitoring system of claim 1, wherein the notification is a visual indicator displayed by the display of the receiver.

14. The analyte monitoring system of claim 13, wherein the visual indicator is a popup screen or an icon.

15. The analyte monitoring system of claim 1, wherein the notification is an audio indicator or a tactile indicator.

16. The analyte monitoring system of claim 15, wherein the tactile indicator comprises a vibrating component in the receiver.

17. The analyte monitoring system of claim 1, wherein the anticipated physiological condition is an elevated analyte concentration.

18. The analyte monitoring system of claim 17, wherein the elevated analyte concentration includes a hyperglycemic condition.

19. The analyte monitoring system of claim 1, wherein the anticipated physiological condition is a depressed analyte concentration.

20. The analyte monitoring system of claim 19, wherein the depressed analyte concentration includes a hypoglycemic condition.

21. The analyte monitoring system of claim 1, wherein the receiver is configured to permit the user to enable or disable the notification.

22. The analyte monitoring system of claim 1, wherein the receiver includes a transceiver configured for bidirectional communication with the sensor electronics initiated by the transmitted command.

23. The analyte monitoring system of claim 22, wherein the transceiver is configured to communicate via a radiofrequency link.

24. The analyte monitoring system of claim 22, wherein the command comprises positioning the receiver within a predetermined distance from sensor electronics.

25. The analyte monitoring system of claim 1, wherein the received signals including multiple data points corresponding to the measured analyte concentration over the predetermined time period includes substantially equally time spaced data points over the predetermined time period.

26. A method, comprising:
transmitting a command to a sensor electronics, wherein the command includes a request for analyte sensor data;
receiving the analyte sensor data from the sensor electronics, the analyte sensor data corresponding to a measured analyte concentration present in a bodily fluid of a user, wherein the received analyte sensor data includes multiple data points corresponding to the measured analyte concentration over a predetermined time period;
displaying the received analyte sensor data from the sensor electronics;
determining a real time analyte concentration level and a current status of an anticipated physiological condition based on the received multiple data points;
displaying an indication of the determined real time analyte concentration level and an indication of the determined current status of the anticipated physiological condition; and
outputting a notification associated with the determined status of the anticipated physiological condition;
wherein the analyte sensor data including the multiple data points corresponding to the measured analyte concentration over the predetermined time period is received in a single data transmission from the sensor electronics in response to the transmitted command.

27. A device for processing analyte sensor data, comprising:
a processor;
a transmitter operatively coupled to the processor and configured to transmit a command to a sensor electronics, wherein the command includes a request for analyte sensor data;
a receiver operatively coupled to the processor and configured to receive the analyte sensor data from the sensor electronics, the analyte sensor data corresponding to a measured analyte concentration present in a bodily fluid of a user, wherein the received analyte sensor data includes multiple data points corresponding to the measured analyte concentration over a predetermined time period;
a display operatively coupled to the processor and configured to display the received analyte sensor data from the sensor electronics; and
a memory including instructions which, when executed by the processor, causes the processor to determine a real time analyte concentration level and a current status of an anticipated physiological condition based on the received multiple data points, display an indication of the determined real time analyte concentration level and an indication of the determined current status of the anticipated physiological condition on the display, and output a notification associated with the anticipated physiological condition;
wherein the analyte sensor data including the multiple data points corresponding to the measured analyte concentration over the predetermined time period is received in a single data transmission from the sensor electronics in response to the transmitted command.
